(19) United States
(12) Patent Application Publication
(10) Pub. No.: US 2026/0207300 A1
(43) Pub. Date: Jul. 23, 2026

(54) GRAVITY-ASSISTED DENTAL DESIGN

(71) Applicant: Modjaw, Villeurbanne (FR)

(72) Inventor: Maxime JAISSON, Porte-De-Savoie (FR)

(73) Assignee: Modjaw, Villeurbanne (FR)

(21) Appl. No.: 19/138,874

(22) PCT Filed: Dec. 14, 2023
(86) PCT No.: PCT/EP2023/085818

§ 371 Date: Jun. 13, 2025

Related U.S. Application Data
(60) Provisional application No. 63/387,656, filed on Dec. 15, 2022.

(51) Int. Cl. A61C 7/00; A61B 34/10; A61B 34/20; A61B 90/00

(52) U.S. Cl. CPC A61C 7/002; A61B 34/10; A61B 34/20; A61B 90/39; A61B 2034/105; A61B 2034/2046; A61B 2090/3983

(57) ABSTRACT

Method for orienting a dental model of a patient with respect to gravity, including: receiving, by a computer system, orientation data; receiving, by the computer system, one or more models of said patient; orienting, by the computer system, said one ore mode models based on said orientation data.

### BACKGROUND

### Field

This disclosure relates generally to dental planning systems and methods. Some embodiments relate to systems and methods for determining patient orientation information using gravitational data.

### Description of the Related Art

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

Dentists typically look for reference planes on the face or head that are likely to be close to the horizon when developing orthodontic or prosthetic treatment plans. Ensuring a proper relationship between the dental arches and the craniofacial references is key to the diagnosis, planning, and execution of treatments that involve adjusting the placement of the patient's teeth, the positioning of prosthetic devices, maxillofacial procedures, and so forth.

Conventionally, dentists locate craniofacial markers determined from anthropometric research to approximate a horizontal plane as closely as possible. The markers used are typically accessible so that they can be recorded and transferred to simulators of a stomatognathic system that includes the dental arches.

Individuals have a natural head position, which can be defined as the natural position of an individual's head when the individual is relaxed, upright, and looking at a distant reference. Several factors affect the natural position of the head, such as visual righting reflexes, airway patency, ethnicity, type of malocclusion, and gravity, among others. Typically, the natural position of the head for a given individual is highly reproducible. The influence of gravity typically causes an individual to orient their head such that the perpendicular to the plane of the lateral semicircular canals is parallel or approximately parallel to the direction of the horizontal reference plane.

### SUMMARY

The systems, methods, and devices described herein each have several aspects, no single one of which is solely responsible for its desirable attributes. Without limiting the scope of this disclosure, several non-limiting features will now be described briefly.

In some aspects, the techniques described herein relate to a method for orienting dental models including: receiving, by a computer system, orientation data; receiving, by the computer system, facial scan data; receiving, by the computer system, one or more dental models, wherein the one or more dental models include at least a maxillary mesh or a mandibular mesh; orienting, by the computer system, the facial scan data based on the orientation data; and registering, by the computer system, the one or more dental models with respect to the facial data. Accordingly, terms like dental models, facial scan data, meshes, etc. may be used indifferently in some of the following embodiments, according to the context, while some of these terms might be used in some other embodiments with a more precise and local meaning.

In some aspects, the techniques described herein relate to a method, wherein the orientation data is associated with the facial scan data.

In some aspects, the techniques described herein relate to a method, wherein the orientation data is associated with photographic data.

In some aspects, the techniques described herein relate to a method, wherein the orientation data is associated with tooth positioning data.

Various combinations of the above and below recited features, embodiments, and aspects are also disclosed and contemplated by the present disclosure.

Additional embodiments of the disclosure are described below in reference to the appended claims, which may serve as an additional summary of the disclosure.

In various embodiments, systems and/or computer systems are disclosed that comprise a computer-readable storage medium having program instructions embodied therewith, and one or more processors configured to execute the program instructions to cause the systems and/or computer systems to perform operations comprising one or more aspects of the above- and/or below-described embodiments (including one or more aspects of the appended claims).

In various embodiments, computer-implemented methods are disclosed in which, by one or more processors executing program instructions, one or more aspects of the above- and/or below-described embodiments (including one or more aspects of the appended claims) are implemented and/or performed.

In various embodiments, computer program products comprising a computer-readable storage medium are disclosed, wherein the computer-readable storage medium has program instructions embodied therewith, the program instructions executable by one or more processors to cause the one or more processors to perform operations comprising one or more aspects of the above- and/or below-described embodiments (including one or more aspects of the appended claims).

### BRIEF DESCRIPTION OF THE DRAWINGS

The following drawings and the associated descriptions are provided to illustrate embodiments of the present disclosure and do not limit the scope of the claims. Aspects and many of the attendant advantages of this disclosure will become more readily appreciated as the same become better understood by reference to the following detailed description, when taken in conjunction with the accompanying drawings, wherein:

FIGS. 1A-1H illustrate examples of orienting photographs according to some embodiments.

FIG. 2 illustrates an example of rotation and cropping according to some embodiments.

FIG. 3 illustrates an example of adjusting the pose of dental capture data according to some embodiments.

FIG. 4 illustrates an example of tracking and orientation hardware that can be used in some embodiments.

FIG. 5 illustrates an example of tracking and measurement hardware according to some embodiments.

FIGS. 6-8 illustrate example embodiments of orientation determination according to some embodiments.

FIGS. 9A-9B illustrates an example of capturing orientation using a reference marker according to some embodiments.

FIG. 10 illustrates an example process flow for determining a gravity vector G in a reference frame of a maxilla tracker according to some embodiments.

FIG. 11 illustrates an example process for collecting and orienting facial and tooth positioning data according to some embodiments.

FIGS. 12-17 illustrate various views of the teeth of a patient according to some embodiments.

FIG. 18 is a block diagram depicting an embodiment of a computer hardware system configured to run software for implementing one or more embodiments disclosed herein.

### DETAILED DESCRIPTION OF CERTAIN EMBODIMENTS

Although certain preferred embodiments and examples are disclosed below, inventive subject matter extends beyond the specifically disclosed embodiments to other alternative embodiments and/or uses and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Overview

As mentioned briefly above, dental practitioners typically consider one or more anatomically-defined reference planes when developing an orthodontic, maxillofacial, or prosthetic treatment plan. For example, a practitioner can measure the Frankfort Horizontal plane, an imaginary reference plane constructed from the porions and the left orbital. Practitioners can additionally or alternatively use other anatomical reference points to approximate a horizontal plane, as will be readily understood by those of skill in the art. While planes based on anatomical points can be useful, there are several drawbacks. Different patients will have somewhat different planes due to differences in anatomy. A horizontal plane defined using anatomical points thus may deviate significantly from true horizontal and can vary significantly from patient to patient. Deviations of the reference plane from true horizontal can have significant impacts on orthodontic treatment planning and can, in some circumstances, result in misdiagnosis. For example, a deviation from horizontal for an anatomically-defined plane (e.g., a Frankfort horizontal plane) can cause a Class III malocclusion to appear to be a Class II malocclusion, which can have significant impacts on treatment. Thus, there is a need for a horizontal reference plane that accurately reflects the true horizontal, rather than relying on approximations based on anatomical points. Accordingly, the consideration of the gravitational axis can be beneficial because, for example, it can enable a dental practitioner to better consider the natural head orientation of the patient (also referred to herein as head carriage) with regard to a true horizontal rather than an approximation that may deviate significantly from the true horizontal, which can lead to improved patient outcomes.

Individuals typically maintain their head in a natural, reproducible orientation with respect to the horizon when they are, for example, walking, having a conversation at sufficient distance, working at a computer with properly set display height, and so forth. For example, during verbal exchanges with sufficient distance between individuals, an individual typically has a natural head carriage that is perceived by others. The teeth can have an overall orientation that is defined by an aesthetic occlusal plane. The aesthetic plane can be defined as a plane that crosses through a midpoint of the bicondylar axis and that is perpendicular to the gravitational axis. In some implementations, the aesthetic plane can alternatively be defined as a plane that crosses through one condyle and that is perpendicular to the gravitational axis. The aesthetic occlusal plane can be defined as a plane that is parallel to the aesthetic plane and which is translated with respect to the aesthetic plane such that it goes through the incisal point. The vestibular surfaces of the maxillary and mandibular teeth can be revealed to other individuals. Preferably, the overall arrangement can be well-aligned with respect to a horizontal plane that is itself perpendicular to the local gravity axis of earth. In some embodiments, the systems and methods herein can offer dental professionals a solution to control the incline of the vestibular surfaces so that the patient can have a smile that satisfies various aesthetic criteria as described herein and/or as known to those of skill in the art. For example, aesthetic criteria can include without limitation smile arc, symmetry, and relative size of the maxillary central incisors, anterosuperior teeth ratio, anterosuperior space, gingival design, level of gingival exposure, buccal corridor, midline and tooth angulation, color, tooth shape, lip volume, and so forth. The evaluation of some aesthetic criteria can depend on the angle of view. Thus, it can be important to analyze aesthetic criteria with respect to a patient's natural head position.

Preferably, when determining how to position a patient's teeth and/or prosthetic device(s), potential surgical interventions (e.g., to alter the forward position of the mandible), and so forth, the patient's natural head orientation (e.g., pitch, yaw, roll) is taken into account. For example, orthodontic, prosthodontic, and/or surgical interventions can be planned while considering the natural orientation of the head with respect to the horizon. If the natural head position is not taken into account, or is taken into account erroneously, for example as a result of reliance on an anatomical reference plane that deviates significantly from a true horizontal plane, it can be difficult and potentially error-prone to produce an aesthetically pleasing result.

Thus, there is a need for systems methods, and devices that can determine the natural head position of a patient. Moreover, it can be important to accurately determine the positions of the teeth, dental arches, and so forth with respect to the patient's head. This can present additional difficulties. For example, a 3D scan of a patient's head can, as discussed in more detail below, often be captured at the same time as orientation data, such that the 3D scan of the patient's head can be readily oriented with respect to gravity. Typically, the teeth, dental arches, and so forth can be captured using dental molds or impressions, intraoral scanners, or other means known to those of skill in the art. As discussed herein, the systems, methods, and devices described in this disclosure can be used to orient the patient's teeth, dental arches, and so forth with respect to gravity, regardless of the particular methods or devices used to obtain an impression or digital representation of the patient's dental arches.

Various systems and methods for locating, recording, and/or displaying lines or planes characterizing the horizontal plane are described herein. These lines, planes, or both can be displayed virtually and shown in relation to the dental arches of the patient. In some embodiments, lines, planes, or both can be positioned by anchoring relative to 2D and/or 3D points of interest that are previously defined. For example, a point of interest can be located on a photo, face scan, bone model, and/or 3D models of the patient's dental arches.

In some embodiments, the horizontal plane can pass through the intercondylar point, which can be a point located equidistant from the two condylar points. In some embodiments, the horizontal plane can be used to define a new coordinate system that can be imposed on a 3D representation of a patient. 3D models can be exported and integrated into movement simulators such as virtual or mechanical articulators. The virtual or mechanical articulator can be configured to reflect the natural orientation of the patient's dental arches.

In some embodiments, the horizontal plane can be configured to pass at the level of the first mandibular molars, for example by taking into account the medio-vestibular cusp, dental point (e.g., inter-incisal point), or both. In some embodiments, a software application can be configured to display the horizontal plane superimposed on a maxillary arch model, mandibular arch model, or both. The maxillary arch model and/or mandibular arch model can be derived from, for example, dental molds or impressions or intraoral scans. For example, when a conventional mold is made of a patient's teeth, the mold can be digitized using a scanner. In some embodiments, the displaying and superimposing can provide an indication of whether the patient's teeth are oriented in a suitable state. In some embodiments, the displaying and superimposing can provide a guideline to a prosthetist or other dental professional when designing new prostheses, when creating a virtual orthodontic treatment plan or device, or both.

Various methods for determining the horizontal plane can be implemented. For example, in some embodiments, one or more sensors that are attached to or part of an imaging device, a tracker (e.g., a facial tracker, forehead tracker, mandible tracker, and so forth), or both can be used to determine orientation information with respect to the direction of gravity or another reference direction, plane, or both. In some embodiments, physical indicators (e.g., reference markers) can be used and can be detected by the imaging device.

In some embodiments, a plane or line perpendicular to terrestrial gravity can be generated, which can be parallel to the horizon. In some embodiments, the generated planes, lines, or both can be used in applying a new coordinate system to 3D models of a patient, to 2D models or images of the patient, and so forth. In some embodiments, 2D imaging techniques can be used to determine one or more lines parallel to the horizon. In some embodiments, 3D imaging techniques can be used to determine one or more planes parallel to the horizon. In some embodiments, multiple 2D views of a patient can be used to define a horizontal plane.

### Inertial Measurement

In aesthetic dentistry, orthodontics, and maxillofacial surgery, photography is often used in the study of the patient and development of a treatment plan, prosthesis, and so forth. Photographic data can often be communicated with a laboratory, with the patient, or both.

Often, imaging equipment used for capturing patient data can include one or more inertial measurement devices, such as a micro electromechanical systems (MEMS) sensor, which can include one or more accelerometers, gyroscopes, and/or magnetometers. In some embodiments, the imaging equipment can, alternatively or additionally, include other hardware that can be used for measuring the orientation of the imaging equipment, such as one or more gyroscopes, accelerometers, geomagnetic sensors, inclinometers, or a combination of sensors.

In some embodiments, a frame captured by a camera can include one or more characteristic elements of features of the face, such as the pupils of the eyes. For example, if the image includes the pupils of the eyes, the interpupillary line can define the orientation of the image. For example, the interpupillary line can be treated as a horizontal line. The frame can be combined with 3D capture data, for example applied as a texture to a 3D model. Such an approach can facilitate the evaluation of aesthetic features of the patient's smile in relation to the anatomical features.

By using orientation data captured using an orientation sensor (e.g., a MEMS sensor), there can be a common reference frame or coordinate system that can facilitate orientation of photos, 3D models of the arches, and so forth.

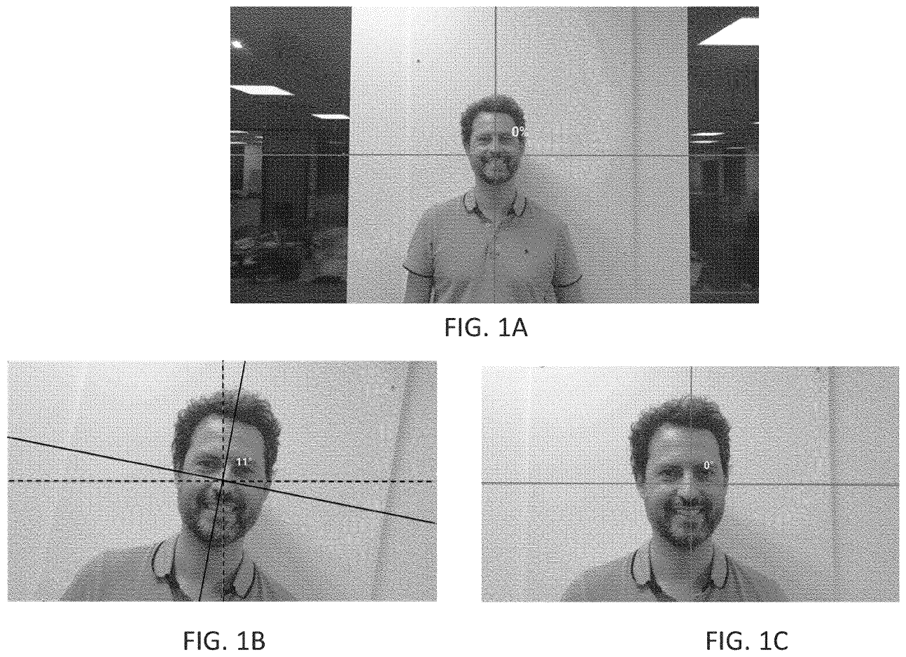

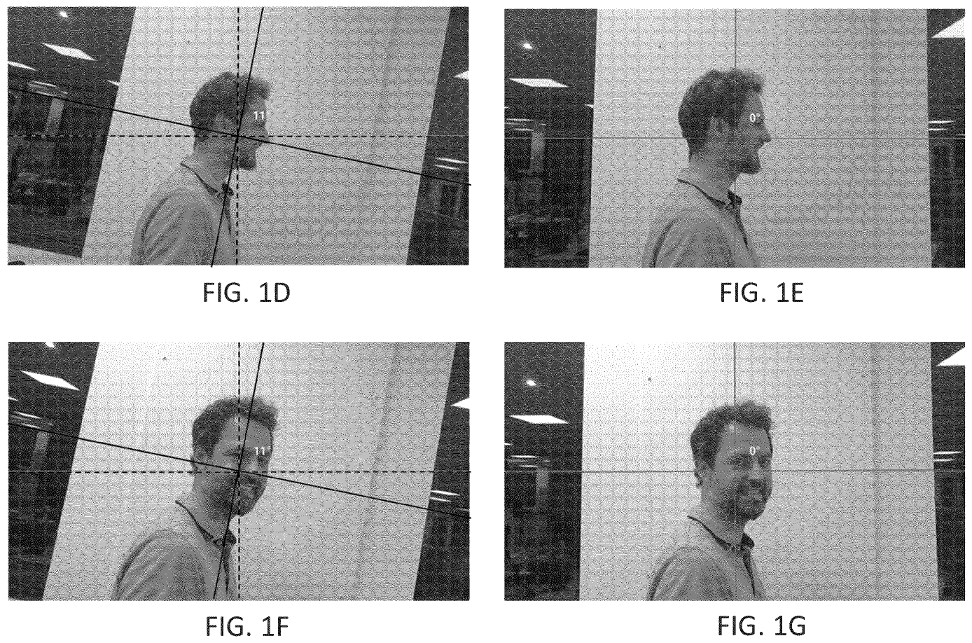

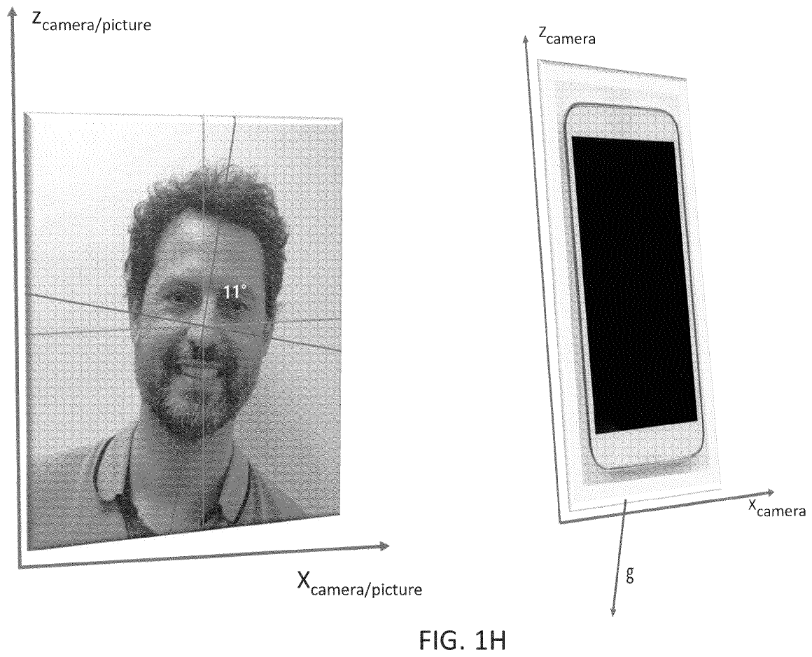

FIGS. 1A-1H illustrate examples of orienting photographs according to some embodiments. For example, FIGS. 1B, 1D, and 1F illustrate an example where the camera frame is tilted by 11 degrees with respect to the gravitational axis. If the camera includes a MEMS sensor or other suitable sensor for determining orientation, the photographs can be readily rotated to correct for the orientation of the camera, as illustrated in FIGS. 1C, 1E, and 1G. In some embodiments, a camera may not include a built-in orientation sensor, but may instead have an orientation sensor fixedly or removably attached to the camera.

As shown in FIG. 1H, in some cases, the camera may be tilted with respect to a vertical axis (e.g., with respect to gravity) and/or in other directions (e.g., the camera may not be pointed head on at the patient, but may instead be angled in one or more directions). Accordingly, alternatively or in addition to rotating an image to correct the horizon (e.g., to make the horizon perpendicular to the direction of gravity), other transformations can be performed. For example, a photograph can skew a patient's head depending on the angle of the camera with respect to the patient. For example, if a camera is tilted downward, the patient's forehead can appear larger relative to the patient's jaw than it actually is. Accordingly, in some embodiments, software can be configured to use the orientation information (and, in some embodiments, additional information such as distance between the camera and patient, focal length, and so forth) to deskew and/or otherwise transform the image to more accurately reflect the patient's true appearance when the patient is maintaining a natural head pose.

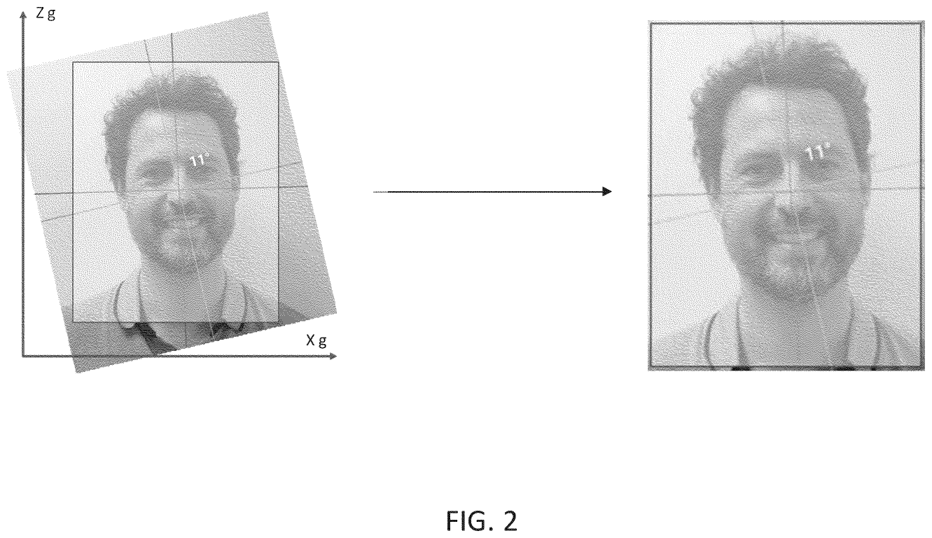

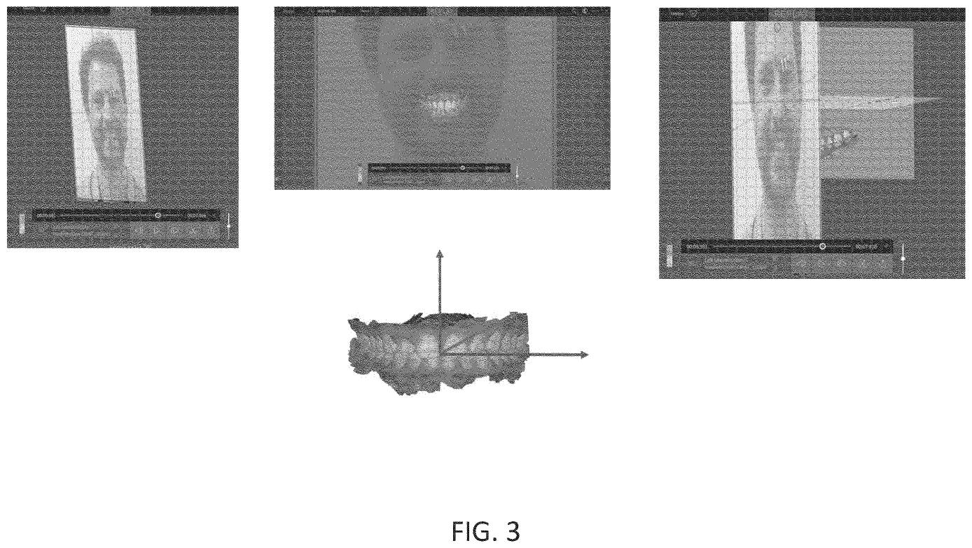

In some embodiments, software can be configured to automatically rotate images based on the determined orientation. In some embodiments, software can be configured to automatically crop images. For example, an image analysis algorithm (which can be, for example, a trained artificial intelligence or machine learning model) can be configured to recognize a face in an image, which can be used to select an area to be cropped. In some embodiments, the software can be configured to extract a rectangular area from an image. In some embodiments, the software can be configured to select a face, for example to extract a face from an image. In some embodiments, the software can be configured to remove a background of the image. FIG. 2 illustrates an example of rotating and cropping an image of a patient's face.

In some embodiments, software can be configured to semi-automatically or manually crop images, rotate images, or both. For example, in some embodiments, the software can be configured to recommend a rotation, crop, or both to a user, and the user can confirm the rotation, crop, or both. In some embodiments, the user can manually rotate and/or crop the image. In some embodiments, the software can be configured to display an indication to the user when the image has been rotated to align the image with the gravitational axis. For example, the software can be configured to display a horizontal line, to outline the image in a different color, or otherwise provide an indication to the user that the image has been rotated an appropriate amount. For example, the software can be configured to determine that a difference between the gravitational axis in the image and the true gravitational axis is less than a threshold amount, which can be, for example, from about 0 degrees to about 5 degrees, from about 0 degrees to about 3 degrees, from about 0 degrees to about 1 degree, or from about 0 degrees to about 0.5 degrees.

In some embodiments, as discussed in more detail below, reference markers can be used to determine orientation, for example if MEMS data is not present. In some embodiments, however, a dedicated marker may not be present. Thus, in some embodiments, an artificial intelligence or machine learning model can be trained to determine a correct image orientation by, for example, recognizing objects or interfaces in the background of the image. For example, if the background includes a shelf, floor-wall interface, wall-ceiling interface, hanging photo, or similar object or interface, the AI/ML model can be trained to orient images such that the object, interface, or both are oriented correctly. For example, the AI/ML model can recognize a shelf and determine an amount of rotation of the image based on the shelf's misalignment from horizontal.

As will be explained in more detail below, the orientation information can also be used to alter the pose of a representation of the patient's teeth to align with an image or 3D scan of a patient's head, as shown in FIG. 3.

In some embodiments, 3D face scans can be captured in a similar manner, for example using a fixed 3D scanner, handheld 3D scanner, smartphone, tablet, etc., that is equipped with an inertial sensor (e.g., a MEMS sensor). The 3D face scan data can have orientation data associated therewith, which can enable the orienting of the 3D face scan data with respect to the direction of gravity. The orientation data can be used to define a horizontal plane. In some embodiments, analysis of the occlusal plane, vestibular surface of the teeth, other aesthetic aspects, and so forth can be carried out with reference to the horizontal plane.

In implementations where the camera, 3D scanner, or other imaging instrument includes an orientation sensor, the camera, 3D scanner, etc., can be used to determine both positioning and orientation data (collectively, pose data). However, in some implementations, as discussed in more detail below, different systems or devices can be used to determine different information. For example, a camera or 3D scanner can be used to obtain positional data, while another sensor or sensors can be used to determine orientation data. For example, images or 3D scan data captured by the camera can be used to determine the relative positions of various features of the patient's head, while an orientation sensor can provide data for determining the orientation of the head with respect to gravity.

While the examples described above relate to imaging without the use of reference markers (also referred to herein as trackers), in some embodiments one or more reference markers can be used when capturing images, 3D face scan data, anchor point data, and so forth. In some embodiments, orientation sensors (e.g., MEMS sensors) can be integrated into the capture device (e.g., a camera such as an infrared camera). In some embodiments, orientation sensors (e.g., MEMS sensors) can be integrated into the one or more reference markers. In some embodiments, each reference marker can include at least one orientation sensor. In some embodiments, not all reference markers may include an orientation sensor. For example, only a portion of the reference markers may include orientation sensors.

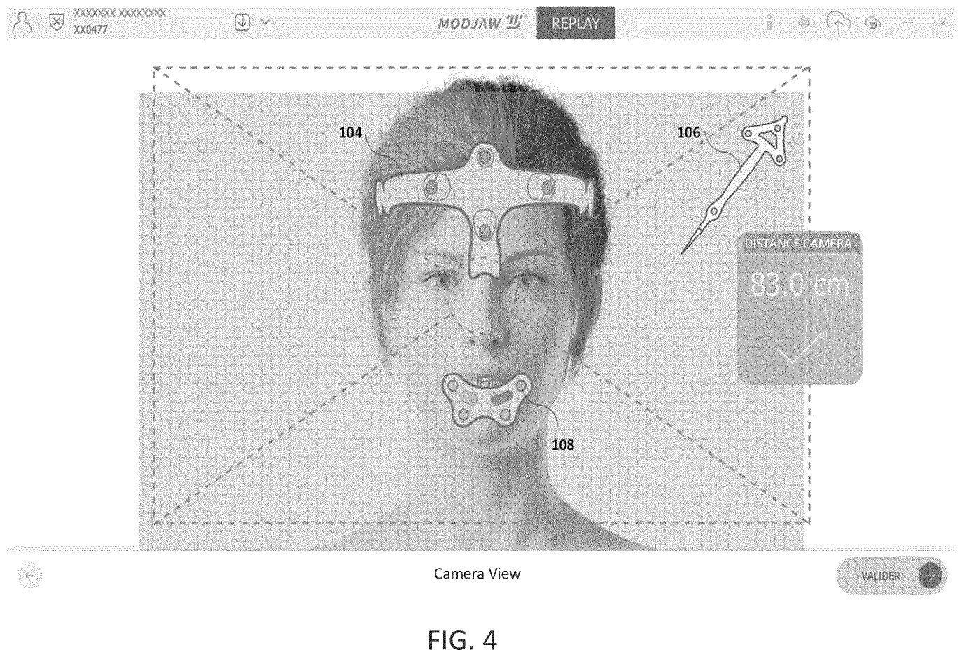

In some embodiments, the patient can be equipped with markers attached at one or more locations on the patient's head. For example, see U.S. Pat. No. 10,265,149, the contents of which are incorporated by reference herein. As discussed therein, a patient can be equipped with a first marker attached to the mandible (mandibular tracker 102) and a second marker (forehead tracker 104) attached to the forehead. In some embodiments, a reference marker 106 can also be captured, for example as shown in FIG. 4. In some embodiments, different and/or additional markers can be used. For example, in some embodiments, a maxillary tracker, which can be broadly similar to the mandibular tracker 102 and forehead tracker 104, but shaped to fit the maxilla of the patient) can be used for tracking the position of the maxilla of the patient.

In some implementations, a pointer (e.g., a pen or stylus) can be used to measure points in the mouth of the patient by means of a pointer having markers detectable by a stereo camera. In some embodiments, an orthonormal reference of 3D models of the dental arches can be associated with an orthonormal reference of the one or more markers affixed to the head of the patient. The markers can be followed in space, for example tracked by a camera such as a stereo camera. The pointer can be used to capture one or more anchor points that can correspond to points included in meshes (dental models) generated from dental impressions or intraoral scans.

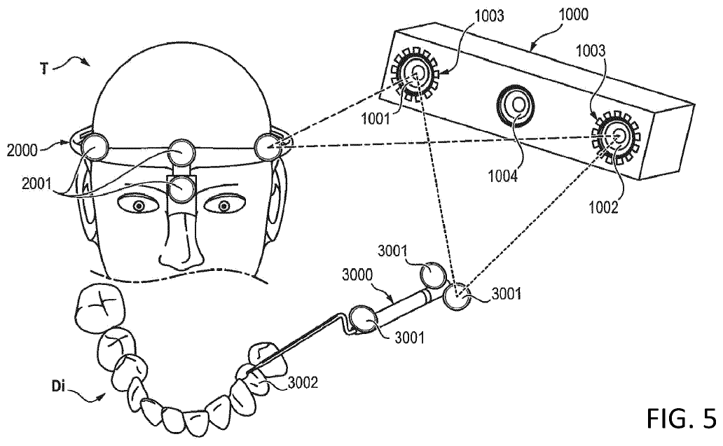

An example pointer, headset, and camera system are depicted in FIG. 5. The system can include a stereoscopic camera 1000 having two objectives 1001, 1002. The camera can include a projector 1004 that can enable the projection of structure light or a laser emitter to project laser bands on the face of the patient. A frontal headset 2000 (e.g., a tiara or forehead tracker) can be placed on the head of the patient. The frontal headset can include a plurality of markers 2001 that can be detected by the stereoscopic camera 1000. The system can include a pointer 3000 having markers 3001 that can be detected by the stereoscopic camera 1000. The stereoscopic camera 1000 can include infrared emitters 1003. The markers 3001 can advantageously comprise an infrared-reflective outer surface. It will be appreciated that while infrared emitters, reflectors, and so forth can be used, other approaches are also possible. For example, the stereoscopic camera 1000 can be configured to detect visible light. In some embodiments, the markers 2001 and markers 3001 can be patterned, colored, or otherwise fashioned such that they can be detected by the stereoscopic camera 1000. Advantageously, in some implementations, the markers 2001 and markers 3001 can be readily distinguished from the face, background, or other features that can be present in captured data.

It will be appreciated that while FIG. 5 illustrates a system having a headset, stereo camera, and pointer, not all components may be present in all implementations. For example, in some embodiments, a head tracker may be used in conjunction with a stereo camera, but a pointer may not be present. In some embodiments, a head tracker may not be used, but a pointer and stereo camera may be used in conjunction with one another to capture the locations (e.g., relative locations) of points of interest in the patient's mouth, on the patient's teeth, or both. In some embodiments, multiple trackers can be used. For example, in some implementations, a patient may be fitted with a first tracker on the patient's forehead and a second tracker on the patient's mandible, as illustrated in FIG. 4.

If the patient is standing or sitting with the head in a natural position while the markers are equipped, the sensors of the camera or other detection instrument can be used to determine the orientation of the trackers in relation to the horizontal plane (e.g., with respect to gravity) when the patient is maintaining a natural head pose. Accordingly, a coordinate system that is related to gravitation can be applied to 3D models of the dental arches, facial 3D models, or any other model of the patient.

In some implementations, trackers can include orientation sensors. In some embodiments, a camera may not include orientation sensing equipment, or it may otherwise be desirable to use orientation sensors included in one or more trackers. In some embodiments, orientation sensors in the trackers can be initially matched to a gravitationally-defined coordinate system, for example during a calibration procedure performed at the factory, by the user (e.g., dental professional), by service technicians, and so forth.

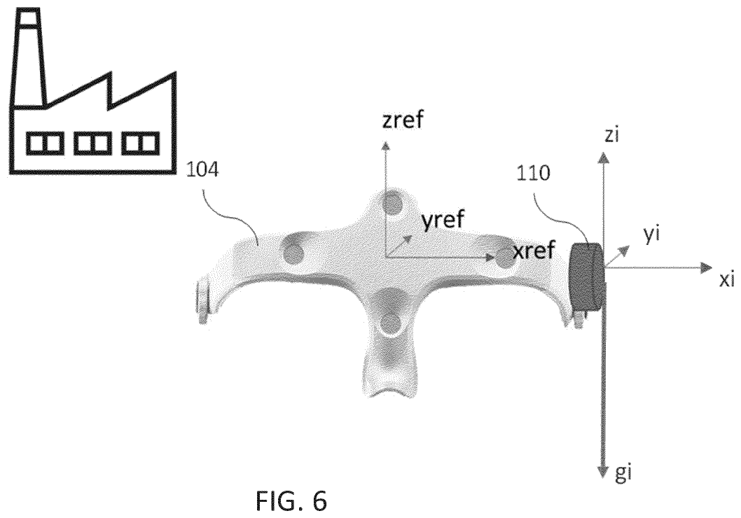

FIG. 6 shows an example embodiment of a forehead tracker 104 having a MEMS sensor 110. As illustrated in FIG. 6, the reference axes for the forehead tracker 104 (e.g., x_{ref}, y_{ref}, and z_{ref}) can be configured with respect to the gravitational axis gᵢ which can be determined using the MEMS sensor 110. For example, z_{ref} can point in the same direction as zᵢ, which can point in a direction opposite the direction of gravity. In a Cartesian coordinate system, the remaining directions x and y can be orthogonal to each other and orthogonal to the z axis. In some embodiments, the reference frame for the forehead tracker 104 can be the same as the reference frame for the MEMS sensor 110 (e.g., xᵢ, yᵢ, zᵢ). While a Cartesian coordinate system is depicted in FIG. 6, it will be appreciated that other coordinate systems are possible. In some embodiments, a right-handed coordinate system can be used, while in other embodiments, a left-handed coordinate system can be used.

In some embodiments, an orientation sensor can be embedded in, screwed to, or removably clipped or otherwise affixed (e.g., with a hook and loop fastener, tape, and so forth) to a tracker. If the orientation sensor is removable, the sensor, the tracker, or both can advantageously be designed such that the orientation sensor can be attached to the tracker in a reproducible manner. For example, a receiving portion of the tracker can be configured (e.g., keyed) such that the orientation sensor can be attached in only one way.

In some embodiments, the orientation sensor(s), whether in a tracker, camera, or both, can be in communication with a computer system, for example via wired or wireless communication, and can be configured to transmit orientation data to the computer system. For example, the sensors can be configured to communicate via Bluetooth, Bluetooth Low Energy, zigbee, or another suitable communication protocol. The orientation data can be used to determine the orientation of the marker with respect to a vertical gravitational axis.

During a scanning procedure, the patient can be asked to place their head in a neutral or natural head position. The orthonormal reference frame can be reset. The orientation sensor(s) can provide the orientation of the trackers (and thereby, the patient's head) with respect to the gravitational axis. 3D models of the patient's dental arches and/or other models can be linked to the tracker orientation. In parallel, a camera (e.g., a stereo camera) can record the positioning of the markers. The orientation of the tracker can be recorded by the camera and, in parallel, the orientation sensor(s) can send information about orientation of the sensor(s) with respect to the gravitational axis. This information can be used to apply a new reference frame or coordinate system to the 3D models (e.g., models of the patient's dental arches, face, and so forth), wherein the new reference frame or coordinate system includes a horizontal plane with respect to gravity.

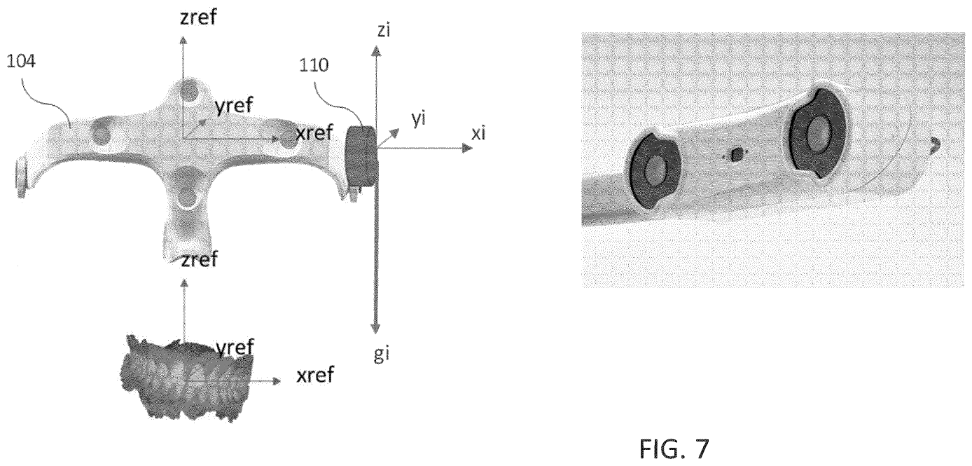

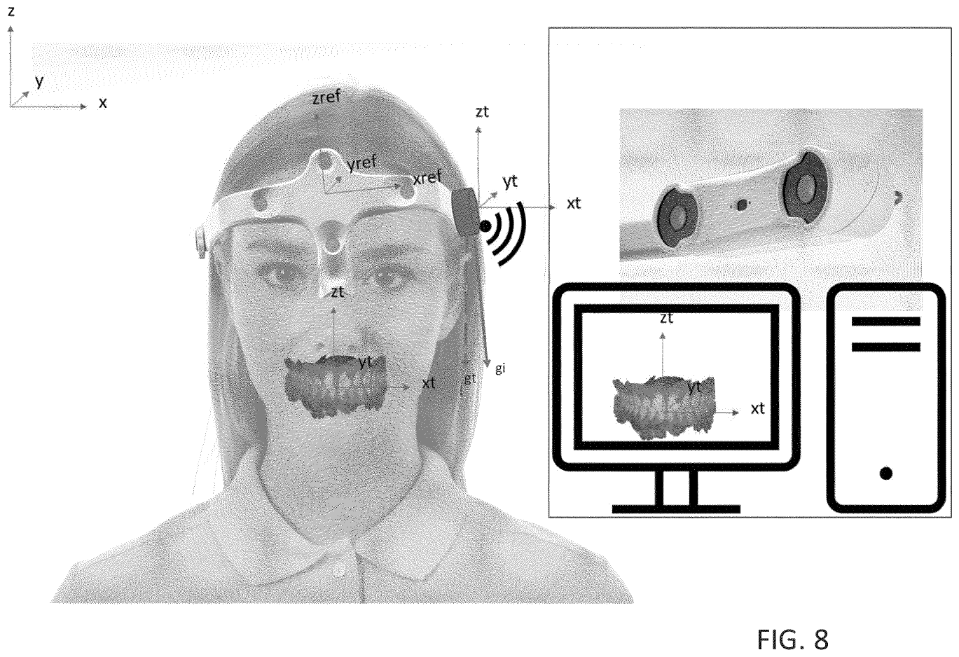

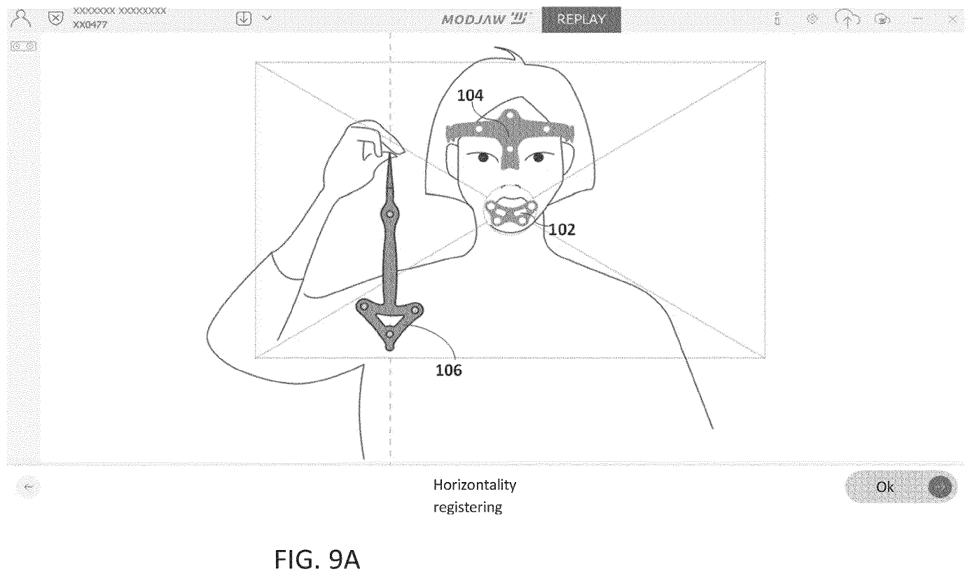

FIGS. 7 and 8 illustrate an example of adjusting pose using orientation information. For example, dental data (e.g., a dental mesh or point cloud) representing a patient's teeth (which can include the mandibular teeth, the maxillary teeth, or both) can be adjusted to align the dental data with the patient's natural head pose.

Preferably, orientation data can be collected at the same time as facial scan data is collected. If both are collected simultaneously, the orientation data can, in some embodiments, be used to correct the scan data to account for movement of the patient during the scanning procedure. However, in some embodiments, orientation and scan data may be collected asynchronously such that the orientation data is not synchronized in time with the facial scan data.

### Orientation Using Computer Vision

While various types of electronic sensors can be used for determining orientation with respect to the gravitational axis, it will be appreciated that such sensors are not necessary. In some embodiments, the gravitational axis can be determined using computer vision algorithms. For example, a reference marker with a known geometric shape, color, pattern, reflective markers, contours, etc. can be placed in the frame of a camera. The object can be suspended from a point such that the object is oriented by the downward force of gravity.

In some embodiments, the orientation of the reference marker with respect to gravity when the reference marker is suspended can be measured separately (e.g., using a camera that is equipped with an orientation sensor) as part of a calibration process. In some embodiments, the reference marker may be designed such that it has a known orientation when under the influence of gravity such that a calibration step may not be included. For example, the reference marker can be an object with a high degree of symmetry, such as a solid of revolution in which the mass of the reference marker is distributed uniformly around an axis of the reference marker, such that when the reference marker is suspended, said axis of the reference marker aligns with the gravitational axis.

The reference marker can be placed in the frame of a camera (e.g., stereo camera, 3D scanner, 2D camera, etc.) during a capture process such that the orientation of the reference marker can be captured at the same time as the capture of the patient. Accordingly, the orientation of the reference marker in the capture data can be used to determine an orientation of the camera and/or the patient's head with respect to the gravitational axis.

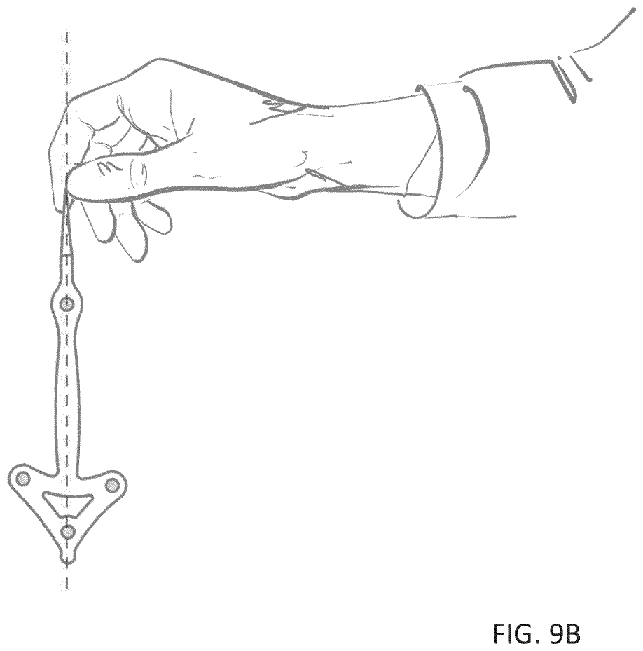

For example, as shown in FIGS. 9A and 9B, the patient can hold the reference marker during the capture process, and the camera can detect the reference marker at the same time as detecting the patient's head, trackers, and so forth. In other implementations, the marker can be suspended, for example from the ceiling or from hardware located above or near the patient.

Alternatively or additionally, the reference marker can be captured before and/or after capturing the patient. For example, the pose of the camera (or other capture device) may not change or may change by a known amount, and thus the orientation of the patient's head with respect to gravity can be determined even if the reference object is captured before or after the patient is captured.

### Determining Orientation

The systems, methods, and devices discussed above can be used to determine the patient's natural head pose. It is also important, as discussed briefly above, to determine the pose of the patient's dental arches (e.g., maxillary arch and mandibular arch) when the patient's head is in its natural position. As discussed below, the orientation data captured above can be applied to the dental arches, even if the dental arch data (e.g., maxillary mesh and mandibular mesh) was collected at a different time and/or without positioning and/or orientation data associated therewith.

A computing system can be configured to, using the orientation data obtained using the methods and systems described above, determine the positioning of the patient's head with respect to horizontal (e.g., with respect to the gravitational axis).

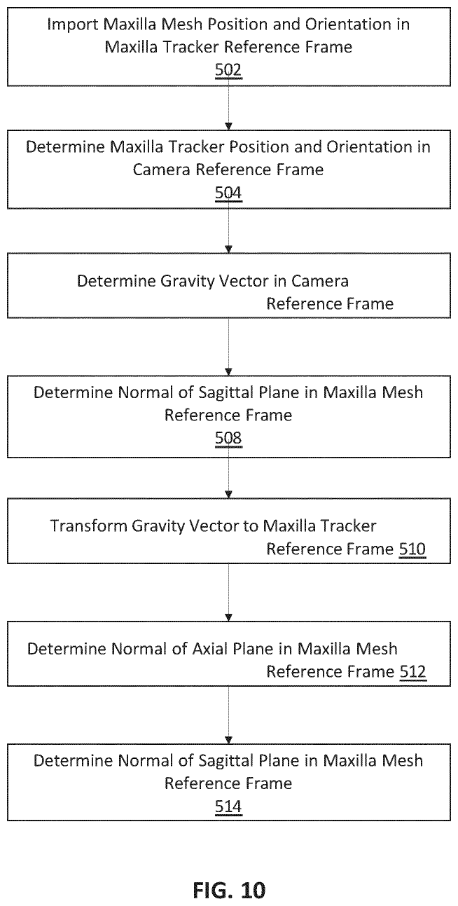

FIG. 10 illustrates an example process flow for determining a gravity vector G in a reference frame of a maxilla tracker. It will be appreciated that other processes could be used, which can include more steps, fewer steps, or steps carried out in an order that is different from the steps depicted in FIG. 10. While FIG. 10 and the following discussion relate to an example process that uses maxilla tracker data, it will be appreciated that the same or a similar process can be used for other trackers, such as mandibular trackers. The process depicted in FIG. 10 can be executed on a computer system, such as a smartphone, desktop, laptop, tablet, server, or other similar device. In some embodiments, the process can be run on a local computer system, such as a laptop, desktop, or tablet located at a dental professional's office or in a laboratory. In other embodiments, the process can be run on a remote system such as a cloud server. For example, data can be transferred or uploaded to a remote server for further processing and/or analysis.

At block 502, the system can import position and orientation data for a maxilla mesh in a maxilla tracker reference frame. At block 504, the system can determine position and orientation data of the maxilla tracker in a camera reference frame (for example, an infrared camera configured to detect trackers such as the maxilla tracker). At block 506, the system can determine a gravity vector G in the camera reference frame. At block 508, the system can determine a normal vector of the sagittal plane in the maxilla tracker reference frame. At block 510, the system can be configured to transform the gravity vector G to a gravity vector G′ in the maxilla tracker reference frame. At block 512, the system can calculate a normal vector of an axial plane in the maxilla tracker reference frame. At block 514, the system can calculate a normal vector of the sagittal plane in the maxilla tracker reference frame. In some embodiments, importing can include applying calculations to orientation data to determine the gravity vector, the normal vector of the sagittal plane, or both.

As one example, position and orientation data (collectively, pose data) can be stored in a 4×4 matrix wherein the matrix elements represent rotations and/or translations. For example, the pose of the imported maxilla mesh in the maxilla tracker reference frame can be represented by a matrix M_{mesh,tracker}. The pose of the maxilla tracker in the camera reference frame can be represented by the matrix M_{tracker,camera}. The gravity vector in the reference frame of the camera can be represented by a vector G. The gravity vector G can be transformed in a vector G′ in the maxilla tracker reference frame by the relation G′=M_{mesh,tracker}⁻¹×M_{tracker,camera}⁻¹×G.

In the example of FIG. 5, there is a tracker reference frame and a camera reference frame. In other embodiments, there can be additional and/or different reference frames. For example, in some embodiments, the maxilla tracker can have its own reference frame and the maxilla mesh can have another, different reference frame. In some embodiments, a new tracker reference frame can be defined (e.g., after identifying the condyles). In some embodiments, the maxilla mesh, mandible mesh, or both can have reference frames (e.g., a maxilla mesh reference frame and/or a mandible mesh reference frame), and a relationship can be determined between a mesh reference frame (e.g., maxilla mesh reference and/or mandible mesh reference frame) and the new tracker reference frame.

Those of skill in the art will readily understand that other implementations are possible. For example, in some embodiments, pose can be represented by a 4×4 matrix of the form

| r₁₁ | r₁₂ | r₁₃ | tₓ |
|---|---|---|---|
| r₂₁ | r₂₂ | r₂₃ | t_{y} |
| r₃₁ | r₃₂ | r₃₃ | t_{z} |
| 0 | 0 | 0 | 1 |

wherein rᵢⱼ (i=1 to 3, j=1 to 3) represent rotations and tₓ, t_{y}, and t_{z} represent translations. In other embodiments, rotations and translations may be represented by different matrices, for example a 3×3 rotation matrix and a translation vector. In some implementations, the rotation matrix can be split into multiple matrices, for example a yaw matrix, a pitch matrix, and a roll matrix. In different embodiments, different coordinate systems can be used, for example Cartesian, spherical, cylindrical, or another coordinate system as may be desirable.

While the above discussion focuses on three dimensional transformations, as discussed above, 2D photographs are commonly used in planning dental or maxillofacial procedures. Thus, in some implementations, matrices appropriate representing 2D position and orientation can be used, such as a 2×2 rotation matrix.

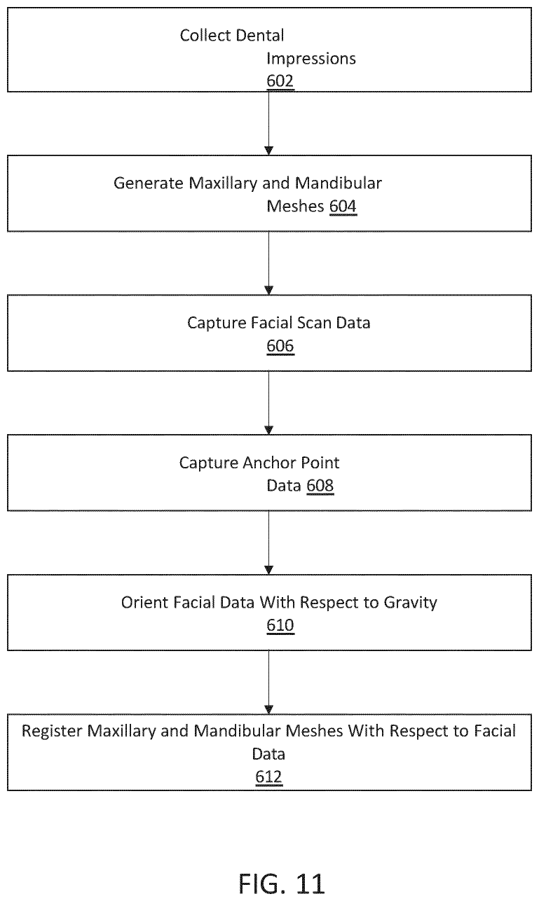

FIG. 11 illustrates an example process for collecting and orienting facial and tooth positioning data according to some embodiments. At block 602, a dental practitioner can collect impressions of the patient's teeth. For example, the practitioner can collect one or more molds or can collect impressions using other means such as an intraoral scanner. At block 604, the molds and/or intraoral scanner data can be used to generate maxillary and mandibular meshes that represent the patient's maxillary and mandibular teeth (and, optionally, additional structure such as part of the gums). At block 606, a practitioner can capture facial scan data. The facial scan data can comprise photographs, 3D scan data, and so forth. More generally, the facial scan data encompasses any digitalization of patient's face, whatever the used technology (photogrammetry, structured light, etc.). At block 608, the practitioner can capture anchor point data, for example using a pointer and camera system, such as the pointer 3000 and stereoscopic camera 1000 depicted in FIG. 5. At block 610, a computer system can be configured to orient the facial data with respect to the gravitational axis, for example using the process depicted in FIG. 10. At block 612, the system can use the anchor point data to register the maxillary and mandibular meshes with respect to the facial scan data.

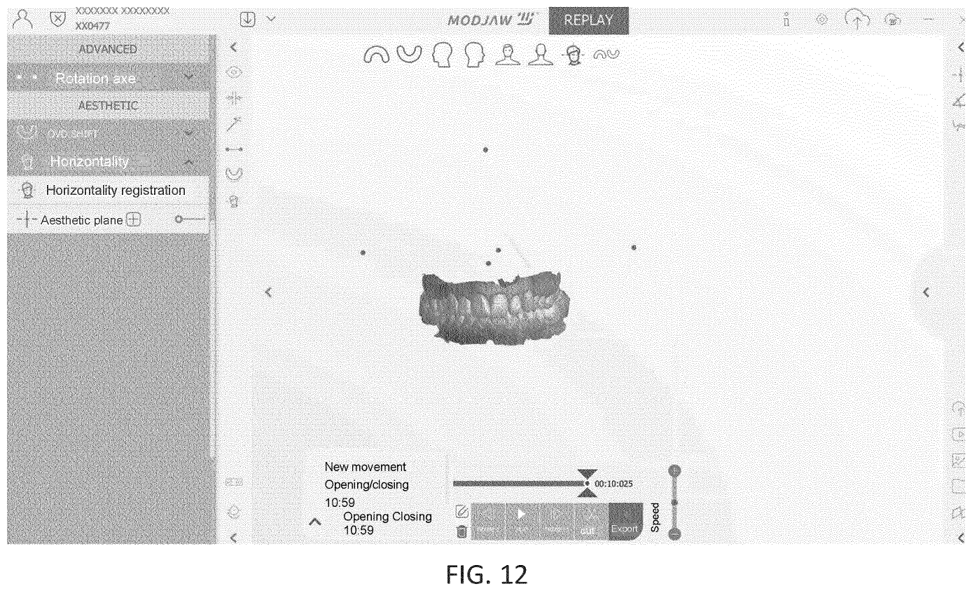

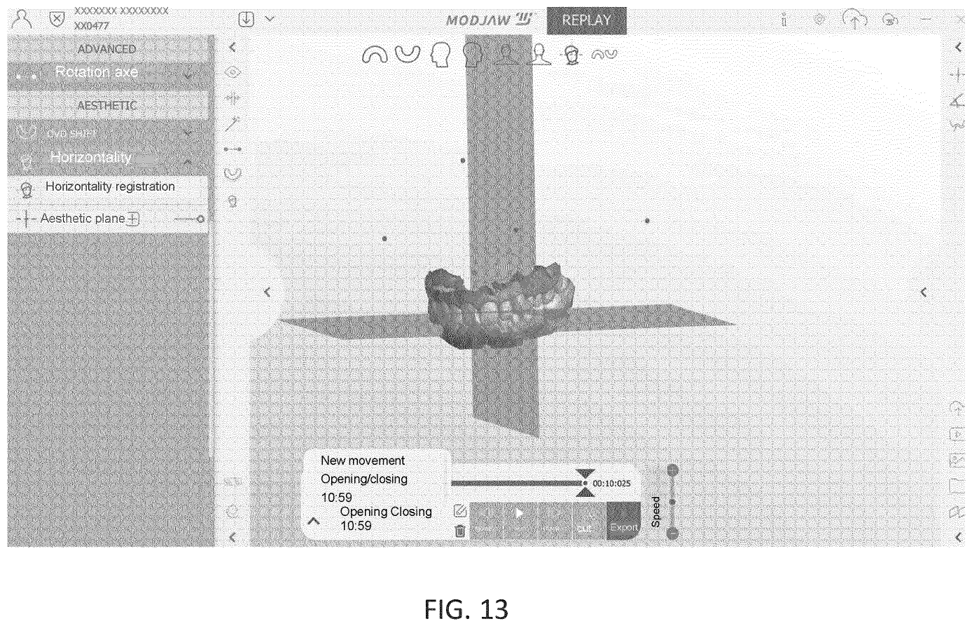

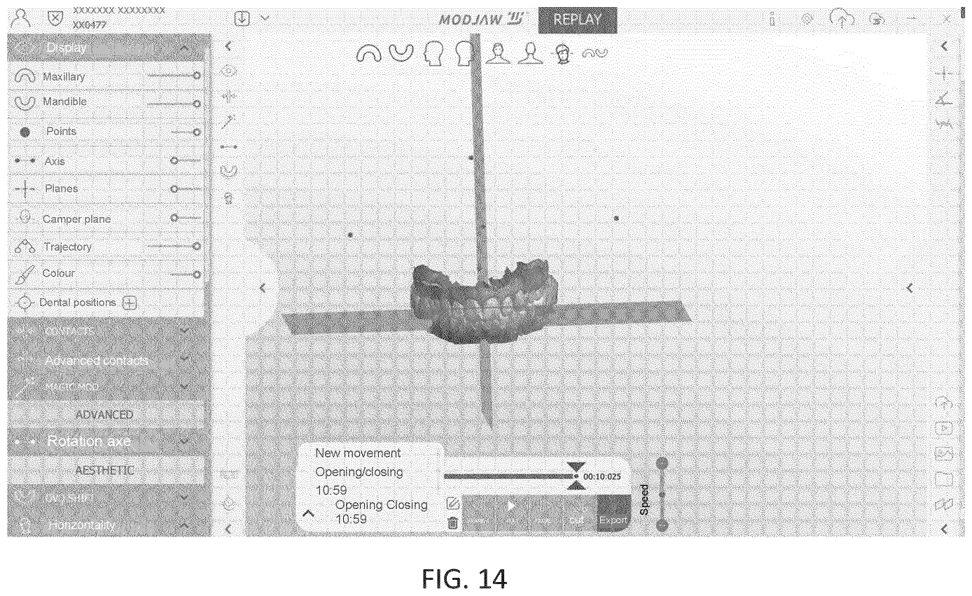

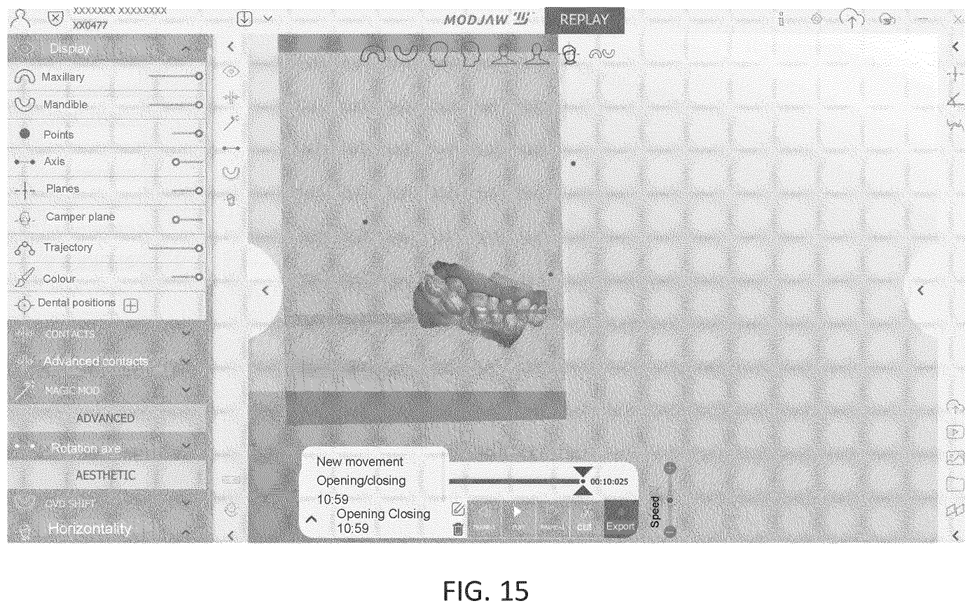

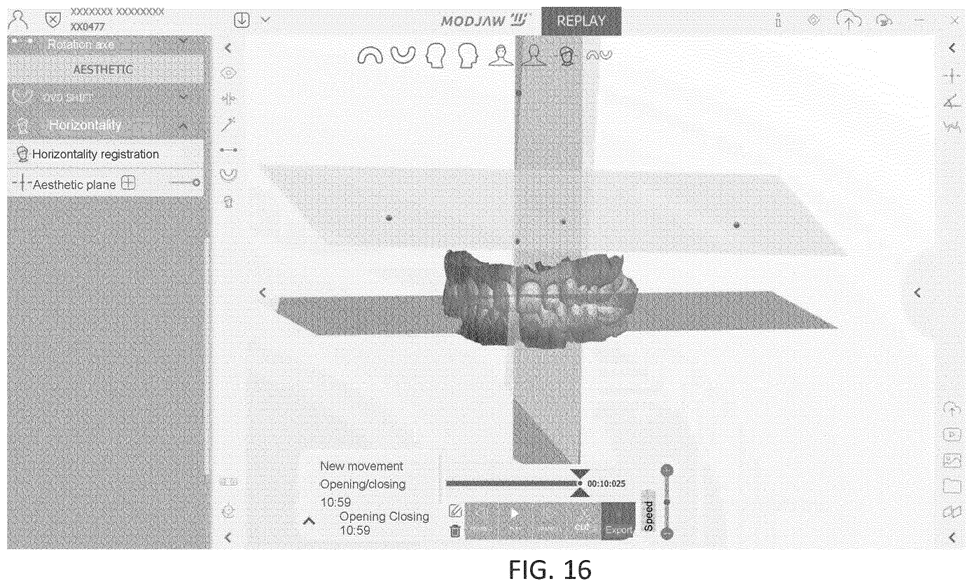

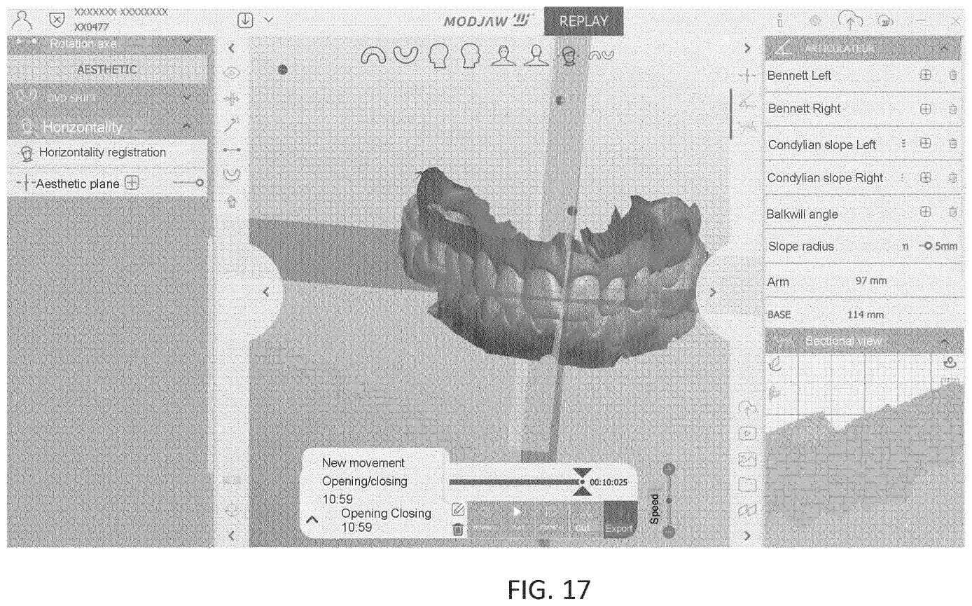

FIGS. 12-17 illustrate various views of the teeth of a patient according to some embodiments. As shown in FIGS. 12-17, planes can be superimposed on the teeth. For example, a horizontal plane can be included in a view of the teeth of the patient. The teeth can be oriented to reflect their natural orientation when the patient is maintaining a natural head pose, as indicated by the blue (original) and red (gravity-assisted) planes in FIGS. 12-17, which can illustrate the reorientation of the teeth using head pose data.

### Computer Systems

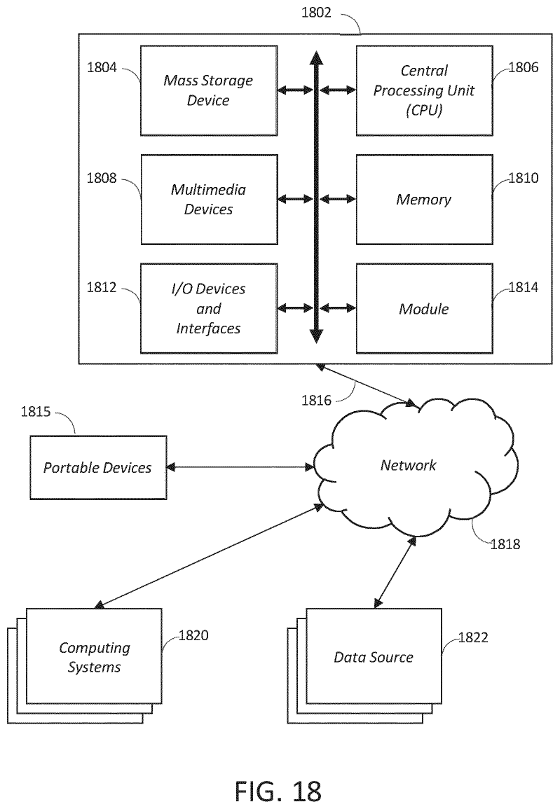

FIG. 18 is a block diagram depicting an embodiment of a computer hardware system configured to run software for implementing one or more embodiments disclosed herein.

In some embodiments, the systems, processes, and methods described herein are implemented using a computing system, such as the one illustrated in FIG. 18. The example computer system 1802 is in communication with one or more computing systems 1820 and/or one or more data sources 1822 via one or more networks 1818. While FIG. 18 illustrates an embodiment of a computing system 1802, it is recognized that the functionality provided for in the components and modules of computer system 1802 may be combined into fewer components and modules, or further separated into additional components and modules.

The computer system 1802 can comprise a module 1814 that carries out the functions, methods, acts, and/or processes described herein. The module 1814 is executed on the computer system 1802 by a central processing unit 1806 discussed further below.

In general, the word “module,” as used herein, refers to logic embodied in hardware or firmware or to a collection of software instructions, having entry and exit points. Modules are written in a program language, such as JAVA, C or C++, Python, or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 1802 includes one or more processing units (CPU) 1806, which may comprise a microprocessor. The computer system 1802 further includes a physical memory 1810, such as random-access memory (RAM) for temporary storage of information, a read only memory (ROM) for permanent storage of information, and a mass storage device 1804, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 1802 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 1802 includes one or more input/output (I/O) devices and interfaces 1812, such as a keyboard, mouse, touch pad, and printer. The I/O devices and interfaces 1812 can include one or more display devices, such as a monitor, that allows the visual presentation of data to a user. More particularly, a display device provides for the presentation of GUIs as application software data, and multi-media presentations, for example. The I/O devices and interfaces 1812 can also provide a communications interface to various external devices. The computer system 1802 may comprise one or more multi-media devices 1808, such as speakers, video cards, graphics accelerators, and microphones, for example.

The computer system 1802 may run on a variety of computing devices, such as a server, a Windows server, a Structure Query Language server, a Unix Server, a personal computer, a laptop computer, and so forth. In other embodiments, the computer system 1802 may run on a cluster computer system, a mainframe computer system and/or other computing system suitable for controlling and/or communicating with large databases, performing high volume transaction processing, and generating reports from large databases. The computing system 1802 is generally controlled and coordinated by an operating system software, such as Windows XP, Windows Vista, Windows 7, Windows 8, Windows 10, Windows 11, Windows Server, Unix, Linux (and its variants such as Debian, Linux Mint, Fedora, and Red Hat), SunOS, Solaris, Blackberry OS, z/OS, iOS, macOS, or other operating systems, including proprietary operating systems. Operating systems control and schedule computer processes for execution, perform memory management, provide file system, networking, and I/O services, and provide a user interface, such as a graphical user interface (GUI), among other things.

The computer system 1802 illustrated in FIG. 18 is coupled to a network 1818, such as a LAN, WAN, or the Internet via a communication link 1816 (wired, wireless, or a combination thereof). Network 1818 communicates with various computing devices and/or other electronic devices. Network 1818 is communicating with one or more computing systems 1820 and one or more data sources 1822. The module 1814 may access or may be accessed by computing systems 1820 and/or data sources 1822 through a web-enabled user access point. Connections may be a direct physical connection, a virtual connection, and other connection type. The web-enabled user access point may comprise a browser module that uses text, graphics, audio, video, and other media to present data and to allow interaction with data via the network 1818.

Access to the module 1814 of the computer system 1802 by computing systems 1820 and/or by data sources 1822 may be through a web-enabled user access point such as the computing systems' 1820 or data source's 1822 personal computer, cellular phone, smartphone, laptop, tablet computer, e-reader device, audio player, or another device capable of connecting to the network 1818. Such a device may have a browser module that is implemented as a module that uses text, graphics, audio, video, and other media to present data and to allow interaction with data via the network 1818.

The output module may be implemented as a combination of an all-points addressable display such as a cathode ray tube (CRT), a liquid crystal display (LCD), a plasma display, or other types and/or combinations of displays. The output module may be implemented to communicate with input devices 1812 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 1802 may comprise a physical or logical connection established between a remote microprocessor and a mainframe host computer for the express purpose of uploading, downloading, or viewing interactive data and databases on-line in real time. The remote microprocessor may be operated by an entity operating the computer system 1802, including the client server systems or the main server system, an/or may be operated by one or more of the data sources 1822 and/or one or more of the computing systems 1820. In some embodiments, terminal emulation software may be used on the microprocessor for participating in the micro-mainframe link.

In some embodiments, computing systems 1820 who are internal to an entity operating the computer system 1802 may access the module 1814 internally as an application or process run by the CPU 1806.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 1802 may include one or more internal and/or external data sources (for example, data sources 1822). In some embodiments, one or more of the data repositories and the data sources described above may be implemented using a relational database, such as Sybase, Oracle, CodeBase, DB2, PostgreSQL, and Microsoft® SQL Server as well as other types of databases such as, for example, a NoSQL database (for example, Couchbase, Cassandra, or MongoDB), a flat file database, an entity-relationship database, an object-oriented database (for example, InterSystems Caché), a cloud-based database (for example, Amazon RDS, Azure SQL, Microsoft Cosmos DB, Azure Database for MySQL, Azure Database for MariaDB, Azure Cache for Redis, Azure Managed Instance for Apache Cassandra, Google Bare Metal Solution for Oracle on Google Cloud, Google Cloud SQL, Google Cloud Spanner, Google Cloud Big Table, Google Firestore, Google Firebase Realtime Database, Google Memorystore, Google MongoDB Atlas, Amazon Aurora, Amazon DynamoDB, Amazon Redshift, Amazon ElastiCache, Amazon MemoryDB for Redis, Amazon DocumentDB, Amazon Keyspaces, Amazon Neptune, Amazon Timestream, or Amazon QLDB), a non-relational database, or a record-based database.

The computer system 1802 may also access one or more databases 1822. The databases 1822 may be stored in a database or data repository. The computer system 1802 may access the one or more databases 1822 through a network 1818 or may directly access the database or data repository through I/O devices and interfaces 1812. The data repository storing the one or more databases 1822 may reside within the computer system 1802.

### Additional Embodiments

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

It will also be appreciated that conditional language used herein, such as, among others, “can,” “could,” “might,” “may,” “for example,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the embodiments are not to be limited to the particular forms or methods disclosed, but, to the contrary, the embodiments are to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (for example, as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (for example, as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles and the novel features disclosed herein.

1. Method for orienting a dental model of a patient with respect to gravity, comprising: receiving, by a computer system, orientation data; receiving, by the computer system, one or more models of said patient; and orienting, by the computer system, said one ore mode models based on said orientation data.

2. The method according to claim 1, wherein said orientation data are captured using an orientation sensor like a micro electromechanical system.

3. The method according to claim 2, wherein said orientation sensor is integrated into a capture device and said orientation data are used to align with an image or 3D scan of the head of said patient.

4. The method according to claim 3, where said capture device is a camera, adapted to capture a frame including one or more characteristic elements of features of the face of the patient.

5. The method according to claim 4, wherein said frame is automatically rotated and/or cropped based on said orientation data.

6. The method according to claim 2, wherein said orientation sensor is included into a tracker adapted to be placed on the head of said patient, and to be in communication with said computer system, and wherein said model is linked to the tracker orientation provided by said orientation sensor.

7. The method according to claim 1, wherein said orientation data are provided by a reference marker designed such that it has a known orientation when under the influence of gravity, which is placed in a frame of a camera during a capture process.

8. The method according to claim 7, wherein said reference marker is a solid of revolution in which the mass is distributed uniformly around an axis of said reference marker, such that when said reference marker is suspended, said axis aligns with the gravitational axis.

9. The method according to claim 7, wherein the orientation of said reference marker is used to determine an orientation of said camera and/or of the head of said patient with respect to the gravitational axis.

10. The method according to claim 1, wherein a scan of the patient's head is captured at the same time of said orientation data.

11. The method according to claim 1, wherein said orientation data are used to define a horizontal plane, said horizontal plane being used to define a coordinate system imposed on a 3D model of said patient, said 3D model being exported and integrated into movement simulators such as virtual or mechanical articulators.

12. The method according to claim 1, wherein the orientation data are associated with photographic data.

13. The method according to claim 1, wherein the orientation data are associated with tooth positioning data.

14. The method according to claim 1, wherein said model is a 3D models of the patient's dental arches.

15. Non transitory computer-readable storage medium having program instructions embodied therewith, the program instructions executable by one or more processors to cause the one or more processors to perform operations according to claim 1.
